(19) United States
(12) Patent Application Publication
(10) Pub. No.: US 2017/0056239 A1
(43) Pub. Date: Mar. 2, 2017

(54) Post-Operative Medical Recovery Device

(71) Applicant: Rolf LANGELAND, Stratford, CT (US)

(72) Inventor: Rolf LANGELAND, Stratford, CT (US)

(21) Appl. No.: 15/176,530

(22) Filed: Jun. 8, 2016

Related U.S. Application Data
(60) Provisional application No. 62/172,331, filed on Jun. 8, 2015.

(51) Int. Cl. A61F 7/00; A61H 9/00; A61H 1/00

(52) U.S. Cl. CPC A61F 7/0085; A61H 1/008; A61H 9/005; A61F 2007/0044; A61F 2007/0042; A61F 2007/0035; A61F 2007/0032; A61F 2007/0056; A61H 2201/0214; A61H 2201/1238; A61H 2205/06; A61H 2205/065; A61H 2205/102; A61H 2205/106

(57) ABSTRACT

A medical recovery device is provided that can be worn by a user following an operation or injury. The medical recovery device may be in the form of a boot to be worn on a foot of the user, which includes one or more windows to provide access to a wound, a cooling system and a compression system to reduce the risk of blood clotting. The medical recovery device may also be in the form of a cast or splint, such as an ankle, knee, wrist or elbow cast.

### CROSS-REFERENCE TO RELATED APPLICATIONS

The present application claims the benefit of U.S. Provisional Patent Application No. 62/172,331 filed on Jun. 8, 2015, which is incorporated by reference in its entirety.

### BACKGROUND OF THE INVENTION

After a medical operation or an injury to a patient's limb or parts thereof, the patient is routinely provided with a boot, cast, splint, or other device that is designed to immobilize, in whole or in part, the injured or operated on area. However, these devices present several drawbacks and medical risks.

First, where there is a wound, such as from a surgical incision, enclosing the wound in such a device can increase the likelihood of an infection or breakdown of the wound. For example, for patients recovering from an operation to the lower leg or foot area, surrounding the incision wound with a short leg cast can increase moisture accumulation and apply excess pressure to the incision wound, thereby increasing the likelihood that the wound will be infected. Further, when a patient is immobilized following an operation or injury, there is an increased risk of blood clotting and deep vein thrombosis occurring in the patient.

Therefore, what is needed is a wearable device that can aid in a patient's recovery after an operation or injury, and which reduces the risk of an infection forming in the injured area and also reduces the risk of blood clots.

### SUMMARY OF INVENTION

The present invention relates to a new and improved medical device to be worn during post-operative recovery, and which addresses the shortcomings of the prior art.

In one embodiment, a medical boot according to the present invention is provided, which is designed to prevent surgical incision pressure and moisture accumulation, which can result in a breakdown of the wound or infection.

In an embodiment of the invention, the boot according to the invention comprises a cooling system. The cooling system may be in the form of an ice cold circulation bladder that is placed inside the boot. The circulation bladder can be connected to an electrical device forming part of the cooling system. The electrical device can be battery powered.

The boot according to the invention may also comprise one or more windows that provide access to a wound. The windows can be hinged or otherwise secured to the boot in a manner that allows the window to be opened temporarily to view the wound, and then closed. In different embodiments of the invention, the size, shape, number and location of the windows can vary.

The medical device according to the invention may also comprise a pump system to prevent blood clotting and deep vein thrombosis (DVT). The pump system may comprise an electrical device connected to a compression bladder in the boot. The compression bladder can be positioned around the forefoot or the calf.

In alternative embodiments of the invention, casts or splints are provided in accordance with the above-described principles of the invention, including ankle, wrist, knee and elbow casts.

According to a first aspect of the invention, a medical recovery boot is provided to be worn by a user following an injury or operation. The medical recovery boot includes a cooling system configured to circulate a cold fluid through the medical recovery boot, at least one window configured to open and provide access to an area on the user, and a compression system configured to increase the user's blood flow.

The medical recovery boot can be made at least in part from a rigid material and is configured to immobilize the user's ankle. The medical recovery boot may further comprise a curved bottom section on the base of the medical recovery boot.

In accordance with an embodiment of the medical recovery boot of the first aspect of the invention, the at least one window is positioned on the medical recovery boot to provide access to a wounded area on the user. The at least one window may comprise a cut-out portion through the medical recovery boot, a cover positioned over the cut-out portion, and means for fastening and unfastening the cover to the medical recovery boot. The means for fastening and unfastening the cover to the medical recovery boot allow the cover to alternate between being unfastened from the boot, at least in part, in order to provide access to the wounded area of the user and being fastened to the medical recovery boot to cover the wounded area of the user. The at least one window may further include a hinge securing a section of the cover to the medical recovery boot, wherein the cover is configured to rotate about the hinge.

Further in accordance with an embodiment of the medical recovery boot of the first aspect of the invention, the cooling system may include a fluid supplying device located externally to the medical recovery boot configured to provide cold fluid, a bladder positioned inside the medical recovery boot configured to circulate the cold fluid provided by the fluid supplying device, and tubing configured to connect the fluid supplying device and the bladder and supply the cold fluid from the fluid supplying device to the bladder. The tubing of the cooling system may comprise a first, inflow tube configured to provide the cold fluid from the fluid supplying device to the bladder and a second, outflow tube configured to circulate the cold fluid from the bladder back to the fluid supplying device. The cold fluid of the cooling system can be water or a solution comprising water and ammonium nitrate. In certain embodiments, the fluid supplying device of the cooling system can be a portable, battery-powered electric device.

Further in accordance with embodiments of the medical recovery boot of the first aspect of the invention, the compression system includes a compression pump and a compression bladder positioned inside the medical recovery boot and attached to the compression pump. The compression system is configured to apply pressure to the user to increase the user's blood flow where the medical recovery boot is worn. In certain embodiments, the compression pump is a portable, battery-powered electric device. In various embodiments of the medical recovery boot according to the first aspect of the invention, the cooling system and the compression system are applied to different locations within the medical recovery boot.

In accordance with certain embodiments of the medical recovery boot according to the first aspect of the invention, the medical recovery boot may comprise means for storing data about the user, wherein the means for storing data is configured to be accessed by a mobile computing device configured to load and display the data.

In accordance with a second aspect of the invention, a medical recovery device is provided to be worn by a user following an injury or operation. The medical recovery device includes a cooling system configured to circulate a cold fluid through the medical recovery device. The medical recovery device also includes a rigid material configured to immobilize an area of the user on which the medical recovery device is worn.

The medical recovery device according to the second aspect of the invention can include a knee cast configured to immobilize the user's knee, a boot or an ankle cast configured to immobilize the user's ankle, a wrist cast or a wrist splint configured to immobilize the user's wrist, and an elbow cast configured to immobilize the user's elbow.

### BRIEF DESCRIPTION OF THE FIGURES

FIG. 1 shows a lateral view of a medical recovery device in the form of a medical boot according to an embodiment of the invention.

FIG. 2 shows an anterior view of the medical recovery device in the form of a medical boot according to an embodiment of an invention.

FIG. 3 shows a lateral view of a medical recovery device in the form of an ankle cast according to an embodiment of the invention.

FIG. 4 shows an anterior view of the medical recovery device in the form of an ankle cast according to an embodiment of the invention.

FIG. 5 shows a medical recovery device in the form of a wrist cast according to an embodiment of the invention.

### DETAILED DESCRIPTION OF THE INVENTION

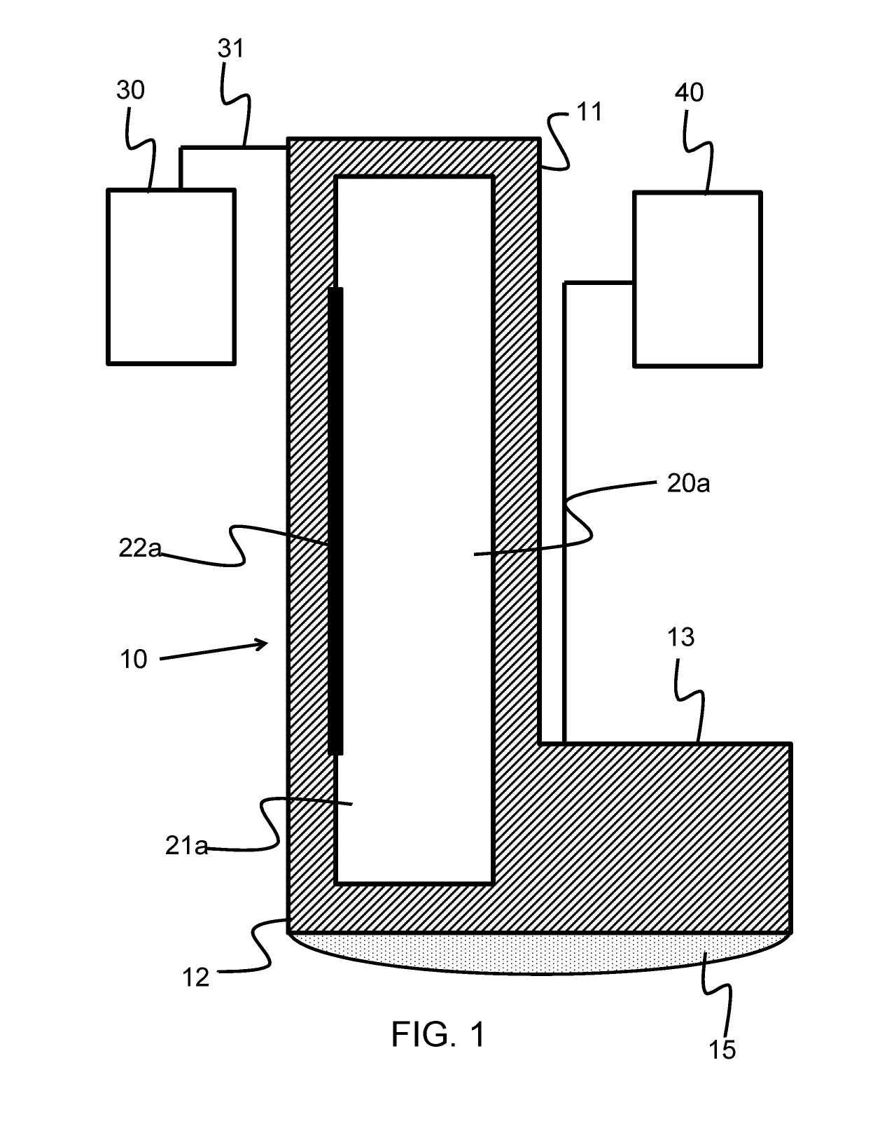

Medical recovery devices according to the present invention will now be described, with reference made to FIGS. 1-5.

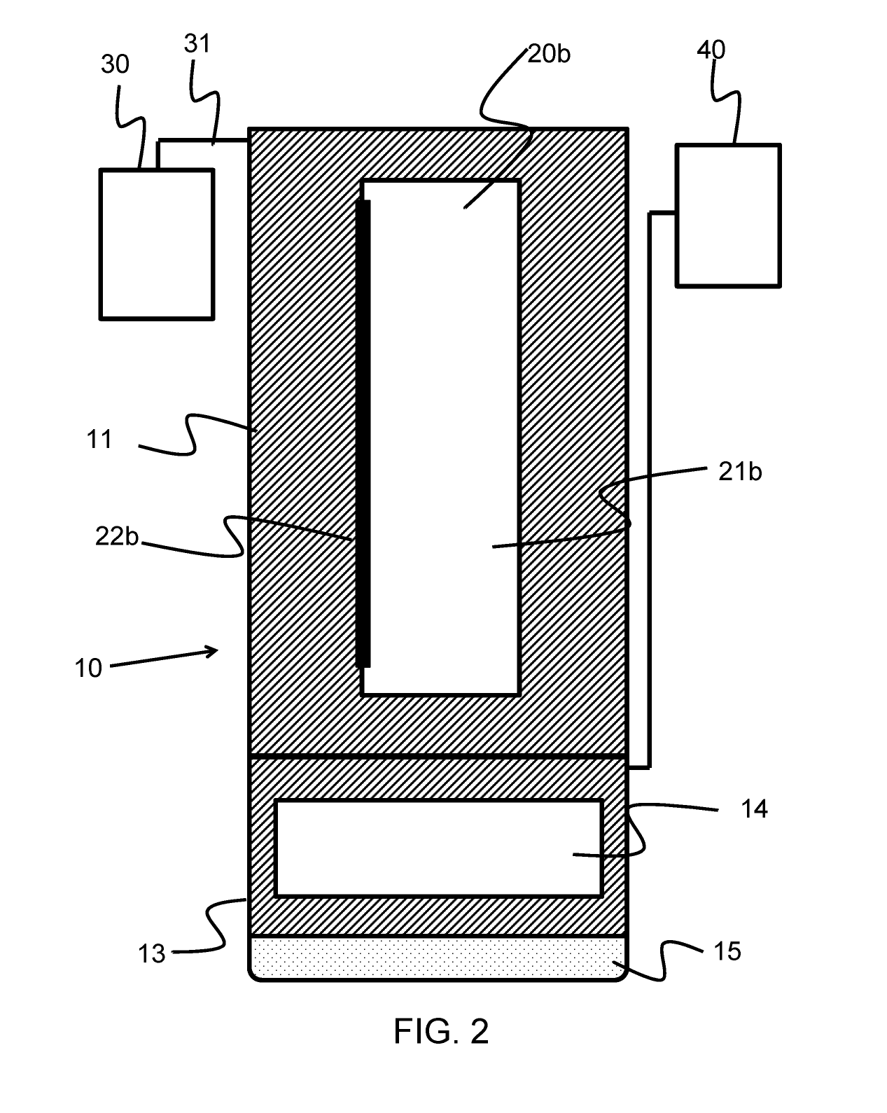

In accordance with an embodiment of the present invention, shown for examples in FIGS. 1-2, a medical boot 10 is provided to decrease moisture and wound breakdown and to reduce infection after an operation in the lower leg, ankle or foot. The boot 10 is preferably made from a rigid material, and has a calf section 11, an ankle section 12 and a forefoot section 13. The boot 10 may also include an opened toe area 14. The boot 10 preferably is configured to entirely or partially immobilize movement of the ankle.

To provide additional comfort for an individual wearing the boot 10, a rocker bottom 15 is provided. The bottom 15 is curved, as shown in FIG. 1 to aid in the in the individual's movement, as he or she plants his or her foot during walking. The bottom 15 can be made from a different and less rigid material than the boot 10.

The boot 10 according to the invention further comprises one or more windows 20 that can be opened to provide a view to the wound. The number and size of the windows 20 that are comprised in the boot 10 can vary. For example, the boot 10 may comprise a window 20a that spans a medial side of the calf section 11 and ankle section 12, and a window 20b spanning the anterior side of the calf section 11. In an alternative embodiment not shown in the Figures, a window can be provided in the posterior of the ankle section 12, to provide access to a wound near the Achilles' tendon. The present invention is not intended to be limited to a particular number, size, shape or position of windows 20 on the boot 10.

The windows 20a, 20b can include covers 21a, 21b positioned over a cut-out portion of the boot 10. The covers 21a, 21b can be attached to the boot on one side or section by hinges 22a, 22b. As a result, the windows 20a, 20b can be easily opened so that the wound area can be accessed and examined. The unhinged sides of the windows 20a, 20b may comprise a fastening and unfastening means, such as adhesives, clasps, hook-and-loop fasteners, snaps or laces, to securely the close the windows 20a, 20b when the wound is not being examined, and subsequently allow opening the windows 20a, 20b for examination of the wound.

The boot 10 further comprises a cooling system. The cooling system may be in the form of an electric-powered device 30 that provides a source of cold water or a cold solution, such as a solution of water and ammonium nitrate, through a tube 31 to a bladder that is positioned inside of the boot 10. The cooling system bladder can be positioned in different locations within the boot 10, such as the calf section 11 or forefoot section 13, depending on where on the lower leg the icing is required. The electrical device 30 can be a portable, battery powered device, so that the user may easily transport the electrical device 30 if necessary, or may be powered through a standard electrical outlet. The electrical device 30 of the cooling system can further be turned off, or disconnected from the boot 10 or tube 31, should the icing no longer be required. The tube 31 includes inflow and outflow tubes, wherein the inflow tubes flows from the electrical device 30 to the boot 10, and the outflow tube flows from the boot 10 back to the electrical device 30. The tube 31 can be in the form of two separate and independent inflow and outflow tubes, or alternatively, can comprise the inflow and outflow tubes partitioned within a single, tubular housing. The boot 10 may include an inlet port configure to detachably receive the inflow tube and an outlet port configured to detachably receive the outflow tube.

The boot 10 can provide continuous circulation of fluid through a cooler which is connected with the inflow/outflow tube 31 connected to the electrical device 30, such as an ice water cooler, when at home resting. The boot 10 can be filled with ice water and the tubes disconnected to allow for travel.

Embodiments of the boot 10 of the invention may also comprise a DVT compression pump 40 to reduce the risk of blood clotting or DVT during recovery. The DVT compression pump 40 may comprise an electrical device connected to a compression bladder that is positioned inside the boot 10. The compression bladder of the DVT compression pump 40 can be positioned in different locations within the boot 10, such as the calf section 11 or forefoot section 13, depending on the location of the injury. The electrical device of the DVT compression pump 40 can be a portable, battery powered device, so that the user may easily transport the electrical device if necessary, or may be powered through a standard electrical outlet. The electrical device of the DVT compression pump 40 can further be turned off or disconnected from the boot 10. Alternative means for applying pressure at the appropriate frequency to stimulate blood flow may also be incorporated into the boot 10.

The cooling system and the compression system can be applied in different locations on the boot 10. For example, the bladder of the cooling system may be positioned in the ankle section 12 of the boot to provide cooling around the ankle while the compression bladder can be located around the forefoot section 13 of the boot. Alternatively, for injuries such as a foot fracture, the bladder of the cooling system may be positioned in the forefoot section 13 of the boot 10 to provide cooling around the foot, while the compression bladder can be located around the calf section 11 of the boot 10.

The boot 10 of the present invention can be arranged to include stylized or design elements. The boot 10 may further comprise in certain embodiments, a bar code, a chip or an RFID (radio frequency identification) tag, or other means for storing data, which is configured to store information amount the patient, including for example information regarding any surgical procedure or the patient's medical history, which can be scanned by a mobile computing device to load the information for viewing by a medical professional.

The boot 10 may also be provided with various text and/or design elements, and in various color schemes, as desired by the user.

Furthermore, in alternative embodiments of the invention, the invention can be in the form of an object other than a boot, while retaining the features of the invention comprised above. For example, the present invention can be in the form of an ankle, knee, wrist or elbow cast comprising the cooling system according to the present invention.

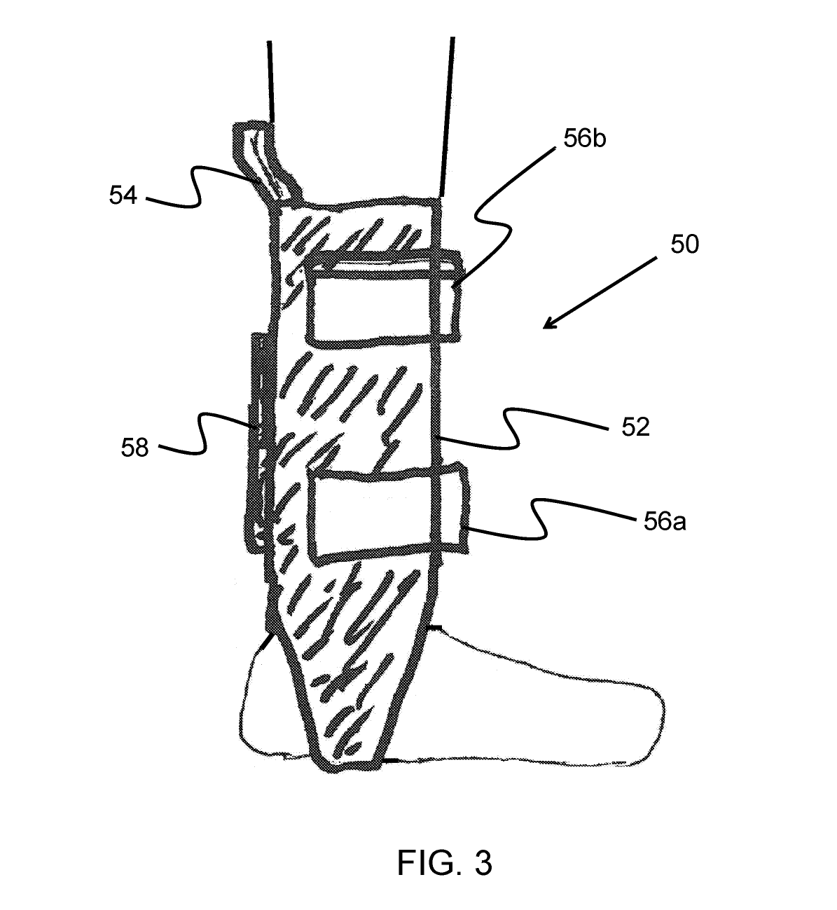

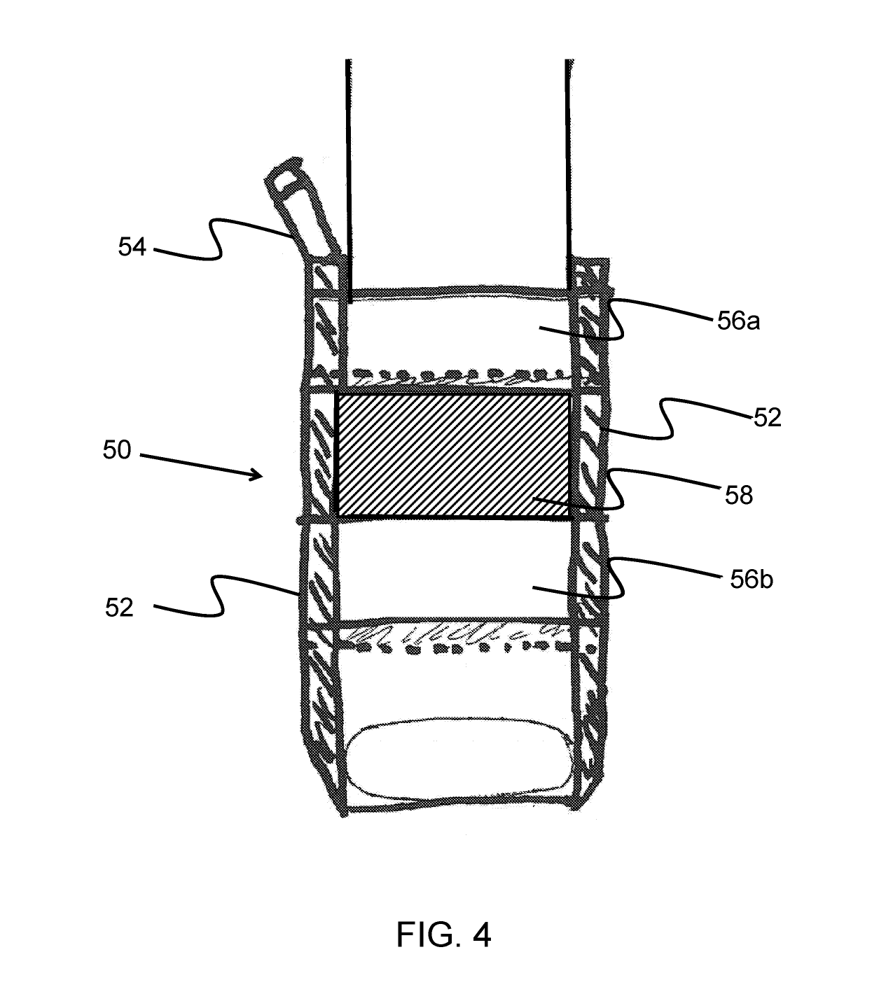

In accordance with an embodiment of the invention, an ankle cast 50 is provided and shown in FIGS. 3 and 4, which includes a cast shell 52 having one or more cast elements extending substantially from the lower calf to the ankle and underneath the foot. The cast shell 52 can be fastened in place using a plurality of fastening elements 56a and 56b. In a preferred embodiment, the fastening elements 56a and 56b include hook-and-loop fasteners; however alternative elements can be used for the fastening elements, such as straps, buckles or adhesive tape for example. The cast shell 52 is not required to extend fully around the leg, and thus for example, the shin can be left uncovered by the cast shell 52, and the use of the fastening elements 56a and 56b can allow the cast 50 to be adjusted and fit multiple sizes of individuals. The cast 50 in this manner can be configured in a similar manner to a standard ankle cast known in the art. The cast 50 further includes a fluid intake port 54 that is secured to an opening in the cast shell 52. Consistent with the embodiments of the invention described above in reference to the boot 10, the fluid intake port 54 is connected to a cooling system (not shown) that provides a cooling fluid that is circulated through the cast shell 52. In certain embodiments of the invention, the cooling fluid can be cold water or a cold solution, including for example a mixture of water and ammonium nitrate. The cast 50 comprises a hinge 58 that is configured to circulate the cooling fluid received through the fluid intake port 54 throughout the cast shell 52, to apply a constant cooling sensation to a person wearing the cast 50. The posterior hinge 58 allows for flow of the fluid from the medial side to the lateral side through the posterior hinge 58.

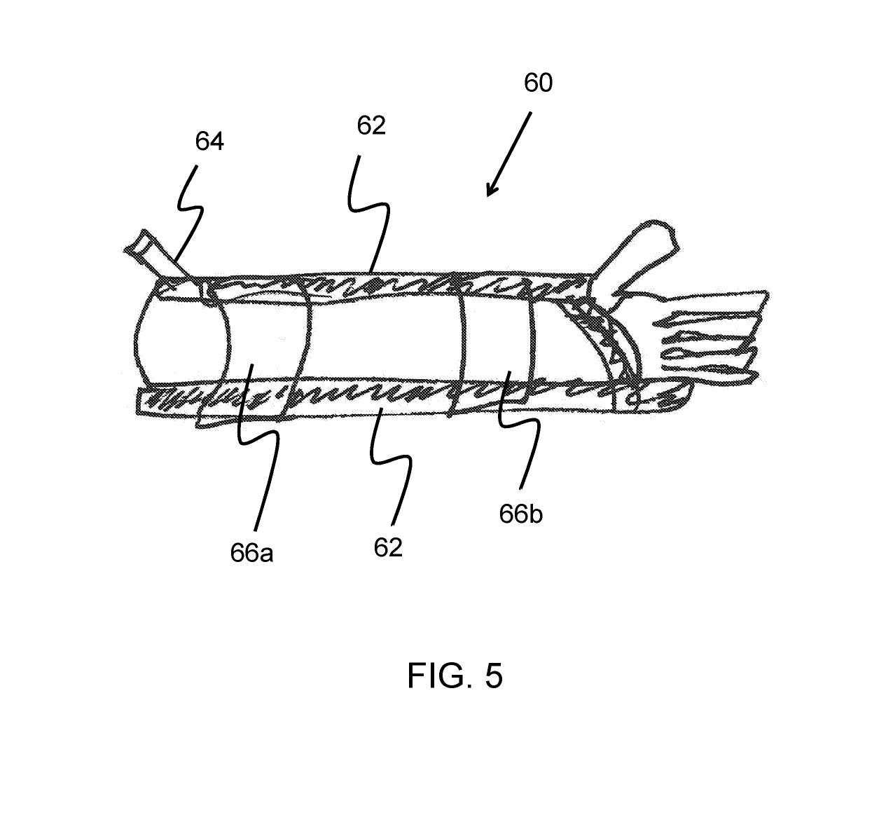

In accordance with a further embodiment of the invention, a wrist cast 60 is provided and shown in FIG. 5, which includes a cast shell 62 having one or more cast elements extending substantially from the wrist to the elbow. The cast shell 62 can be fastened in place using a plurality of fastening elements 66a and 66b. In a preferred embodiment, the fastening elements 66a and 66b include hook-and-loop fasteners; however alternative elements can be used for the fastening elements, such as straps, buckles or adhesive tape for example. The cast shell 62 is not required to extend fully around the leg, and thus for example, the forearm can be left uncovered by the cast shell 62, and the use of the fastening elements 66a and 66b can allow the cast 60 to be adjusted and fit multiple sizes of individuals. The cast 60 in this manner can be configured in a similar manner to a standard wrist cast known in the art.

The cast 60 further includes a fluid intake port 64 that is secured to an opening in the cast shell 62. Consistent with the embodiments of the invention described above in reference to the boot 10, the fluid intake port 64 is connected to a cooling system (not shown), and provides a cooling fluid that is circulated through the cast shell 62. In certain embodiments of the invention, the cooling fluid can be cold water or a cold solution, including for example a mixture of water and ammonium nitrate. The cast 60 is configured to circulate the cooling fluid received through the fluid intake port 64 throughout the cast shell 62, to apply a constant cooling sensation to a person wearing the cast 60.

Generally, the casts or splints 50 or 60 can be configured in one of two versions. In the first version, a freezer-type splint or cast is provided, wherein ammonium nitrate or other cooling fluid is present within a section of padding, which is placed in a freezer to cool, and then put on after cooling. In the second version, which is similar to the design of the boot 10, cooler inflow/outflow tubes are provided which connect the casts or splints 50 or 60 with an iced cooler.

The casts in accordance with the present invention may further comprise features found in the boot 10 described with respect thereto, but not shown in the casts 50 or 60 of FIGS. 3-5. Examples of such embodiments will be described further with reference made to FIGS. 3-5, including for example, a compression system and/or one or more windows as described herein.

While there have been shown and described and pointed out fundamental novel features of the invention as applied to preferred embodiments thereof, it will be understood that various omissions and substitutions and changes in the form and details of the devices and methods described may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice.

What is claimed:

1. A medical recovery boot configured to be worn by a user and comprising:
a cooling system configured to circulate a cold fluid through the medical recovery boot;
at least one window configured to open and provide access to an area on the user; and
a compression system configured to increase the user's blood flow.

2. The medical recovery boot according to claim 1, wherein the medical recovery boot is made at least in part from a rigid material and is configured to immobilize the user's ankle.

3. The medical recovery boot according to claim 2, further comprising a curved bottom section on the base of the medical recovery boot.

4. The medical recovery boot according to claim 1, wherein the at least one window is positioned on the medical recovery boot to provide access to a wounded area on the user.

5. The medical recovery boot according to claim 4, wherein the at least one window comprises:
a cut-out portion through the medical recovery boot;
a cover positioned over the cut-out portion; and
means for fastening and unfastening the cover to the medical recovery boot, such that the cover can alternate between being unfastened from the boot, at least in part, in order to provide access to the wounded area of the user and being fastened to the medical recovery boot to cover the wounded area of the user.

6. The medical recovery boot according to claim 5, wherein the at least one window further comprises a hinge securing a section of the cover to the medical recovery boot, wherein the cover is configured to rotate about the hinge.

7. The medical recovery boot according to claim 1, wherein the cooling system comprises:
a fluid supplying device located externally to the medical recovery boot configured to provide cold fluid;
a bladder positioned inside the medical recovery boot configured to circulate the cold fluid provided by the fluid supplying device; and
tubing configured to connect the fluid supplying device and the bladder and supply the cold fluid from the fluid supplying device to the bladder.

8. The medical recovery boot according to claim 7, wherein the tubing of the cooling system comprises a first, inflow tube configured to provide the cold fluid from the fluid supplying device to the bladder and a second, outflow tube configured to circulate the cold fluid from the bladder back to the fluid supplying device.

9. The medical recovery boot according to claim 7, wherein the cold fluid is water.

10. The medical recovery boot according to claim 7, wherein the cold fluid is a solution comprising water and ammonium nitrate.

11. The medical recovery boot according to claim 7, wherein the fluid supplying device is a portable, battery-powered electric device.

12. The medical recovery boot according to claim 1, wherein the compression system comprises:
a compression pump; and
a compression bladder positioned inside the medical recovery boot and attached to the compression pump;
wherein the compression system is configured to apply pressure to the user to increase the user's blood flow where the medical recovery boot is worn.

13. The medical recovery boot according to claim 12, wherein the compression pump is a portable, battery-powered electric device.

14. The medical recovery boot according to claim 1, further comprising means for storing data about the user, wherein said means for storing data is configured to be accessed by a mobile computing device configured to load and display said data.

15. The medical recovery boot according to claim 1, wherein the cooling system and the compression system are applied to different locations within the medical recovery boot.

16. A medical recovery device configured to be worn by a user and comprising:
a cooling system configured to circulate a cold fluid through the medical recovery device; and
a rigid material configured to immobilize an area of the user on which the medical recovery device is worn.

17. The medical recovery device according to claim 16, wherein the medical recovery device is a knee cast configured to immobilize the user's knee.

18. The medical recovery device according to claim 16, wherein the medical recovery device is an ankle cast configured to immobilize the user's ankle.

19. The medical recovery device according to claim 16, wherein the medical recovery device is a wrist cast or a wrist splint configured to immobilize the user's wrist.

20. The medical recovery device according to claim 16, wherein the medical recovery device is an elbow cast configured to immobilize the user's elbow.
